(19) United States
(12) Patent Application Publication
(10) Pub. No.: US 2025/0000714 A1
(43) Pub. Date: Jan. 2, 2025

(54) SMART DIAPER EMBEDDED WITH WETNESS TRIGGERED SENSORIAL BATTERY AND WIRELESS COMMUNICATION SYSTEM

(71) Applicants: ARIZONA BOARD OF REGENTS ON BEHALF OF ARIZONA STATE UNIVERSITY, Scottsdale, AZ (US); HANGZHOU VANTRONICS BIOTECHNOLOGY CO. LTD., Hangzhou, Zhejiang (CN); HANGZHOU MIHENG INTELLIGENT BIOMEDICAL TECHNOLOGY CO LTD., Hangzhou, Zhejiang (CN); VANTRONICS (HANGZHOU) INTELLIGENT TECHNOLOGY CO. LTD., Hangzhou, Zhejiang (CN)

(72) Inventors: Hanqing JIANG, Chandler, AZ (US); Wei ZENG, Mesa, AZ (US); Haigang ZHAO, Shenzhen, Guangdong (CN); Wenwen XU, Hangzhou, Zhejiang (CN); Chulei ZHONG, Zhejiang Province (CN); Lei MANG, Zhejiang Province (CN)

(21) Appl. No.: 18/690,511

(22) PCT Filed: Sep. 10, 2021
(86) PCT No.: PCT/CN2021/117689

§ 371 Date: Mar. 8, 2024

(51) Int. Cl. A61F 13/42

(52) U.S. Cl. CPC A61F 13/42; A61F 2013/424

(57) ABSTRACT

Methods, systems, and apparatus, including wetness monitoring systems comprising a smart diaper comprising: a sensorial battery comprising at least two electrodes; and a transmitter, wherein the sensorial battery is configured to provide electric current to the transmitter in the presence of water or urine, and wherein the transmitter is configured to emit a signal comprising a unique identifier when the electric current is received from the sensorial battery; and a computing device comprising: a receiver; a user interface; one or more processors; and a computer-readable storage device coupled to the one or more processors and having instructions stored thereon which, when executed by the one or more processors, cause the one or more processors to perform operations comprising: receiving, via the receiver, the signal emitted by the transmitter, and providing, via the user interface, an indication that the smart diaper is wet.

### BACKGROUND

Diapers are used not only for babies, but also for individuals who are incapable of controlling their bladder or bowel movements or unwilling to use the toilet. These individuals include people with medical conditions such as people who are bedridden, or in a wheelchair. However, diapers have a major drawback, they cause skin rash because the skin is exposed to prolonged wetness, increasing skin power of hydrogen (pH) caused by urine which may result in breakdown of the stratum corneum. It is therefore important to reduce the time that the moisture is in contact with the skin by periodically replacing the diaper. Moreover, it is difficult to check the state of the diaper in babies and the elderly without removing their clothes. Nurses and other caregivers spend a lot of time performing this task. Optimizing this process can therefore help to reduce the cost of health services.

### SUMMARY

Early products or devices for incontinence detection are moisture detectors, wetness signaling devices or wet diaper detectors. These devices use standard electronic components such as a sensor/detector, transmitter, receiver, battery or power supply, and alarm. Several ideas of incorporating extra functionality into diapers have also been developed. A battery is employed to power the transmitter in these simple moisture detectors or wetness signaling devices. These batteries are often hard, which make babies or elderly persons uncomfortable.

Accordingly, embodiments of the present disclosure are generally directed to systems for determining a physical environmental condition from a remote location. More particularly, embodiments of the present disclosure are directed to a wetness monitoring system employed to identify a wet diaper by embedding a sensorial battery inside the diaper.

In some embodiments, the described wetness monitoring system provides an economical and long-range solution by integrating inactive and self-powered sensorial battery with wireless transmission system that can be discarded with the wet diaper. In some embodiments, the sensorial battery is formed by conductive electrodes and a dried separator comprising metallic salts. In some embodiments, a wireless transmitter is sealed and integrated with the sensorial battery inside the diaper. In some embodiments, a wireless transmitter is disposed outside the diaper and sealed and integrated with the sensorial battery that is disposed inside the diaper.

In some embodiments, a remote device such as multiple function receiver, a smart personal device, or a display, is employed to communicate with the embedded wireless transmitter to announce a wet diaper condition. For example, a multiple function receiver can identify any of several diapers in a day care center. As another example, several strategically located receivers may be employed in a hospital with each capable of recognizing and reporting a wet diaper within its range to a central computer.

In one aspect, disclosed herein, are wetness monitoring systems comprising: a smart diaper comprising: a sensorial battery comprising at least two electrodes; and a transmitter, wherein the sensorial battery is configured to provide electric current to the transmitter in the presence of water or urine, and wherein the transmitter is configured to emit a signal comprising a unique identifier when the electric current is received from the sensorial battery; and a computing device comprising: a receiver; a user interface; one or more processors; and a computer-readable storage device coupled to the one or more processors and having instructions stored thereon which, when executed by the one or more processors, cause the one or more processors to perform operations comprising: receiving, via the receiver, the signal emitted by the transmitter, and providing, via the user interface, an indication that the smart diaper is wet. In some embodiments, the sensorial battery is embedded on an interior surface of the smart diaper. In some embodiments, the smart diaper comprises an absorption layer. In some embodiments, the sensorial battery is embedded within between 2 and 20 millimeters of the absorption layer of the diaper. In some embodiments, the sensorial battery comprises an electrochemical cell. In some embodiments, the sensorial battery, when wet, derives electrical energy from spontaneous redox reactions taking place within the electrochemical cell. In some embodiments, the at least two electrodes comprise a positive and a negative electrode. In some embodiments, the sensorial battery comprises a separator that mechanically isolates the positive and negative electrodes. In some embodiments, the separator comprises dried filter paper that has absorbed a metallic salt solution. In some embodiments, the separator is configured to release the dried metallic salt solution in the presence of water or urine as an ionized liquid to electrically connect the at least two electrodes generating the electric current via a potential difference and charge flow. In some embodiments, at least one of the electrodes comprises copper. In some embodiments, at least one of the electrodes comprise zinc. In some embodiments, the signal is emitted as a wireless beacon by the transmitter. In some embodiments, the computing device comprises a smart phone or smart tablet. In some embodiments, the operations comprise: determining, based on the unique identifier, a user associated with the smart diaper, and providing, via the user interface, an indication that user requires a new smart diaper. In some embodiments, the operations comprise: before determining the user, receiving information associating the smart diaper to the user via the unique identifier; and persisting the association between the smart diaper and the user.

In another aspect, disclosed herein, are smart diapers comprising: a smart tag embedded in an interior surface of a diaper, the smart tag comprising: a transmitter; and a sensorial battery comprising at least one anode, at least one cathode, and a separator comprising dried filter paper that has absorbed a metallic salt solution, the separator mechanically isolating the at least one anode from the at least one cathode, wherein the separator is configured to release the dried metallic salt solution in the presence of water or urine as an ionized liquid to electrically connect the at least one anode and the at least one cathode generating electric current, wherein the sensorial battery is configured to provide the electric current to the transmitter, and wherein the transmitter is configured to emit a signal comprising a unique identifier when the electric current is received. In some embodiments, the smart diaper comprises an absorption layer. In some embodiments, the sensorial battery is embedded within between 2 and 20 millimeters of the absorption layer of the diaper. In some embodiments, the at least one cathode comprises copper. In some embodiments, the at least one anode comprises zinc. In some embodiments, the sensorial battery comprises an alkaline battery, an aluminum-air battery, a Bunsen cell, a chromic acid cell, a Clark cell, a Daniell cell, a Galvanic cell, a Grove cell, a Leclanché cell, a magnesium battery, a mercury battery, a nickel oxyhydroxide battery, a paper battery, a silver-oxide battery, a Voltaic pile, a zinc-air battery, a zinc-carbon battery, or a zinc chloride battery. In some embodiments, the signal is emitted as a wireless beacon by the transmitter.

In another aspect, disclosed herein, are computer-implemented methods for monitoring the wetness of a smart diaper. These methods are executed by one or more processors and comprise: receiving, from a transmitter embedded in a smart diaper, a signal comprising a unique identifier associated with the smart diaper, the transmitter powered through a sensorial battery configured to provide electric current to the transmitter in the presence of water or urine; determining, based on the unique identifier, a user associated with the smart diaper, and providing, via a user interface, an indication that a user requires a new smart diaper. In some embodiments, the methods comprise: before receiving the signal, scanning, via a scanning device, a barcode comprising the unique identifier; receiving, via an input device, user data regarding the user and instructions to associate the user with the smart diaper; and persisting the association between the smart diaper and the user. In some embodiments, the scanning device comprises a camera. In some embodiments, the input device comprises a touch screen.

Particular implementations of the subject matter described in this disclosure can be implemented so as to realize one or more of the following advantages. In some embodiments, the described wetness monitoring system is economical and employs an inactive power source and becomes active when the wetness triggers the inactive power source. In some embodiments, the described wetness monitoring system can be used to identify specific wet diapers in locations (e.g., a hospital ward, a childcare center, and so forth) where a plurality of individuals are in close relation to one another wear diapers that need to be monitored and precisely identified.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1A depicts a perspective schematic view of a non-limiting, exemplary embodiment of a smart diaper;

FIG. 1B depicts a non-limiting, exemplary embodiment of the sensorial battery;

FIG. 2 depicts a non-limiting, example environment that can be employed to execute implementations of the present disclosure:

FIGS. 3A-3B depict graphs showing the voltage and current curves after adding urine to a pair of sensorial batteries;

FIG. 4 depicts a non-limiting, exemplary embodiment of a sensorial-battery system with four pairs of batteries;

FIGS. 5A-5B depict graphs showing the voltage and current curves after adding urine to four pairs of sensorial battery;

FIG. 6 depict a non-limiting, exemplary of a barcode that can be employed in the described wetness monitoring system;

FIG. 7 depicts a table that includes non-limiting examples of metallic salts that can be employed to form a wetness sensitive separator;

FIG. 8 depicts a flow diagram of a non-limiting, exemplary process or monitoring the wetness of a smart diaper; and

FIG. 9 depicts a non-limiting exemplary computer system that can be programmed or otherwise configured to implement methods or systems of the present disclosure.

### DETAILED DESCRIPTION

Before any embodiments of the disclosure are explained in detail, it is to be understood that the disclosure is not limited in its application to the details of embodiment and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical or hydraulic connections or couplings, whether direct or indirect.

It should also be noted that a plurality of hardware and software-based devices, as well as a plurality of different structural components may be used to implement the implementations. In addition, implementations may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one implementation, the electronic based aspects of the disclosure may be implemented in software (for example, stored on non-transitory computer-readable medium) executable by one or more processors. As such, it should be noted that a plurality of hardware and software-based devices, as well as a plurality of different structural components may be utilized to implement various implementations. It should also be understood that although certain drawings illustrate hardware and software located within particular devices, these depictions are for illustrative purposes only. In some implementations, the illustrated components may be combined or divided into separate software, firmware and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

Embodiments of the present disclosure are generally directed to systems for determining a physical environmental condition from a remote location. More particularly, embodiments of the present disclosure are directed to a wetness monitoring system employed to identify a wet diaper by embedding a sensorial battery inside the diaper.

Accordingly, described herein, in certain embodiments, are wetness monitoring systems comprising: a smart diaper comprising: a sensorial battery comprising at least two electrodes; and a transmitter, wherein the sensorial battery is configured to provide electric current to the transmitter in the presence of water or urine, and wherein the transmitter is configured to emit a signal comprising a unique identifier when the electric current is received from the sensorial battery; and a computing device comprising: a receiver; a user interface; one or more processors; and a computer-readable storage device coupled to the one or more processors and having instructions stored thereon which, when executed by the one or more processors, cause the one or more processors to perform operations comprising: receiving, via the receiver, the signal emitted by the transmitter, and providing, via the user interface, an indication that the smart diaper is wet.

Also described herein, in certain embodiments, are smart diapers comprising: a smart tag embedded in an interior surface of a diaper, the smart tag comprising: a transmitter; and a sensorial battery comprising at least one anode, at least one cathode, and a separator comprising dried filter paper that has absorbed a metallic salt solution, the separator mechanically isolating the at least one anode from the at least one cathode, wherein the separator is configured to release the dried metallic salt solution in the presence of water or urine as an ionized liquid to electrically connect the at least one anode and the at least one cathode generating electric current, wherein the sensorial battery is configured to provide the electric current to the transmitter, and wherein the transmitter is configured to emit a signal comprising a unique identifier when the electric current is received.

Also described herein, in certain embodiments, are computer-implemented methods for monitoring the wetness of a smart diaper. These methods are executed by one or more processors and comprise: receiving, from a transmitter embedded in a smart diaper, a signal comprising a unique identifier associated with the smart diaper, the transmitter powered through a sensorial battery configured to provide electric current to the transmitter in the presence of water or urine; determining, based on the unique identifier, a user associated with the smart diaper, and providing, via a user interface, an indication that a user requires a new smart diaper.

### Definitions

Unless otherwise defined, all technical terms used herein have the same meaning as commonly understood by one of ordinary skill in the art to which the present subject matter belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “real-time” refers to transmitting or processing data without intentional delay given the processing limitations of a system, the time required to accurately obtain data and images, and the rate of change of the data and images. In some examples, “real-time” is used to describe the presentation of information obtained from components of embodiments of the present disclosure.

As used herein, a sensorial battery includes an electrochemical cell that provides a voltage and currents when wet. In some embodiments, a sensorial battery is connected to electrical loads such as a smart tag.

As used herein, a smart tag includes an electric circuit and an antenna.

As used herein, a smart diaper includes a sensorial battery and a smart tag embedded within a diaper. In some embodiments, when the diaper becomes wet, the sensorial battery provides power to the smart tag, which then wirelessly transmit a signal to a network.

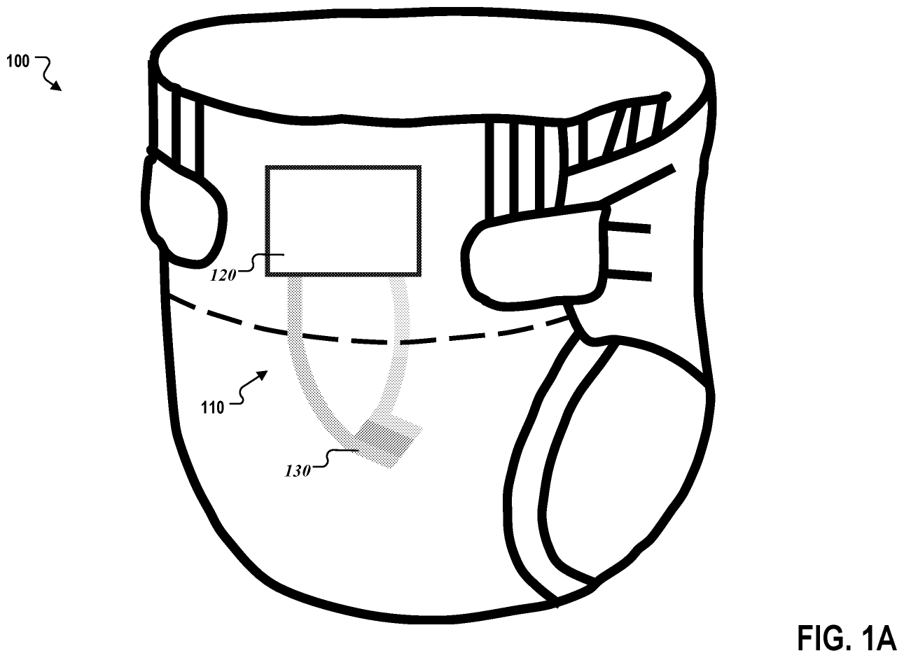

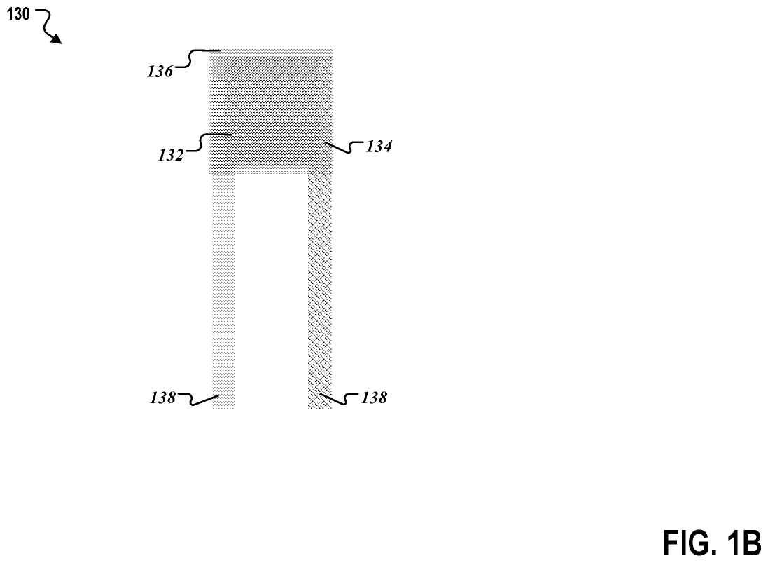

FIG. 1A depicts a perspective schematic view of an exemplary embodiment of a smart diaper 100. As depicted, the smart diaper 100 includes an embedded smart tag 110. The smart tag 110 includes a transmitter 120 and a sensorial battery 130. In some embodiments, the sensorial battery 130 is arranged on an interior surface of the smart diaper 100. In some embodiments, the sensorial battery 130 is arranged on an interior surface of the smart diaper 100 in proximity (e.g., in range between a few (2-3) millimeters to about twenty millimeters) to an absorption layer (e.g., an interior layer) of the diaper.

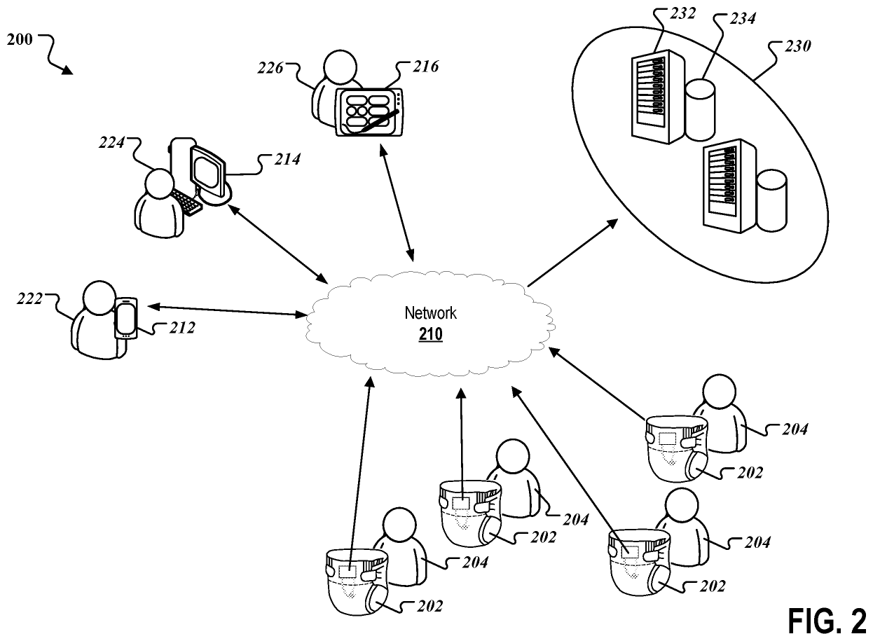

In some embodiments, the transmitter 120 is configured to emit a wireless signal when an electric current is received from the sensorial battery 130. For example, as depicted in FIG. 2, the transmitter 120 may broadcast the signal to a monitor, smart device, wireless gateway, back-end server, network service, and the like. In some embodiments, when powered, the transmitter 120 transmits a wireless beacon. In some embodiments, the transmitter 120 is a component of a transceiver.

In some embodiments, the sensorial battery 130 is employed to detect wetness by generating an electric current when that battery is wet. In some embodiments, the sensorial battery 130 derives electrical energy from spontaneous redox reactions taking place within the electrochemical cell.

FIG. 1B depicts an exemplary embodiment of the sensorial battery 130. As depicted, the sensorial battery 130 includes two electrodes 132 and 134 and a wetness sensitive separator 136. In some embodiments, the separator 136 is positioned between the electrodes 132 and 134. In some embodiments, an ionized liquid, such as CuSO4, is formed between the electrodes 132 and 134 when the separator 136 becomes wet. In such embodiments, the ionized liquid electrically connects the electrodes 132 and 134 and reacts with the anode or cathode materials that comprise the electrodes 132 and 134 to generate a potential difference and charge flow to power the transmitter 120, as shown in FIG. 1A. In some embodiments, the electrodes 132 and 134 and separator 136 are substantially rectangular-shaped. In some embodiments and as depicted, the sensorial battery 130 includes terminals 138 that connect to transmitter.

In some embodiments, the electrodes 132 and 134 are positioned parallel relative to each other on the opposite surfaces of the separator 136. In some embodiments, the electrodes 132 and 134 are positioned at an angle relative to each other. In some embodiments, one of the electrodes 132 and 134 is thicker and/or longer than the other electrode. In some embodiments, the electrodes 132 and 134 comprise various types of materials including, but not limited to, metals, metal oxides, electrically conductive polymers or any suitable materials that can derive electrical energy from spontaneous redox reactions.

In some embodiments, the separator 136 is configured to mechanically and electrically isolate the electrodes 132 and 134, while allowing ionic exchange to occur between the electrodes and the formed electrolyte. In one embodiment, the separator 136 provides both a barrier to block the positive and negative electrodes 132 and 134 as well as a wetting layer to keep the respective sensorial battery wet with electrolyte, thus allowing ionic exchange. In some embodiments, the separator 136 comprises a microporous membrane. Generally, any microporous membrane that is ionically conductive may be employed as the separator 136. For example, a polyolefin-based separator having a porosity of between about 30 and 80 and an average pore size of between about 0.005 and 0.3 micron. In some embodiments, the separator 136 comprises microporous filter paper. For example, cellulose filters from Whatman®. Other separator materials, such as nylon-based materials and microporous polyolefins (e.g., polyethylenes and polypropylenes), and so forth, known in the art may be employed to form the separator 136.

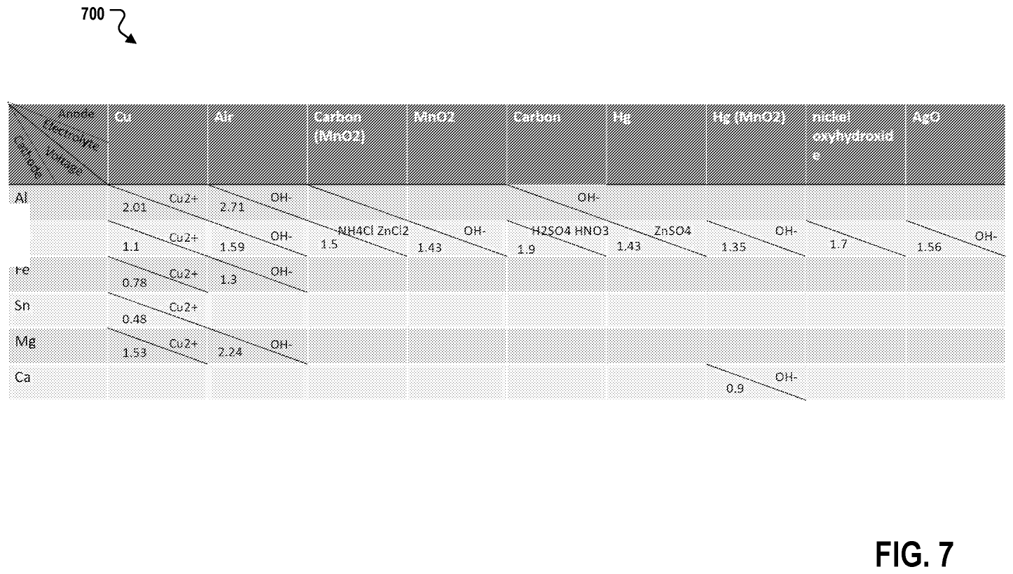

In some embodiments, the separator 136 comprises filter paper and a metallic salt solution that has been dried or crystallized. For example, the filter paper is immersed in a metallic salt solution (e.g., CuSO4 solution) and dried (e.g., air dried or on a hot plate). Various types of metallic salts may be employed to form the separator 136. For example, CuSO4, copper chloride (CuCl2), sodium hydroxide (NaOH) and so forth. FIG. 7 depicts a table 700 that includes metallic salts that can be employed to form the wetness sensitive separator 136 depending on the pairing of the electrodes 132 and 134.

In some embodiments, when the separator 136 becomes wet with a liquid (e.g., water, urine), the metallic salt ionizes. In such embodiments, the ionized liquid or electrolyte reacts with the cathode or anode materials that form the positive and negative electrodes 132 and 134 to generate a current and power wireless signal emission via the transmitter 136.

In some embodiments, the structure of the sensorial battery 130 includes a substrate, which may be formed from a sheet of flexible material on which the electrode 132 and 134 and the separator 136 are fixed. In some embodiments, the substrate comprises Polyester (PET) film and includes holes to allow liquid (e.g., water or urine) to flow throw the battery wetting the separator 136.

As an example, to form the sensorial battery 130, a cathode (e.g., copper) electrode is pasted on the flexible substrate using double-sided tape. Subsequently, a separator 136 that includes dried metallic salts and is larger than the cathode electrode is placed on the cathode electrode. Finally, an anode (e.g., zinc) electrode is fixed on the separator 136, forming a layer-by-layer structure.

The principle of establishing a potential difference across the electrodes 132 and 134 is explained below. The voltage (electromotive force E°) produced by, for example, a copper Cu-Zinc (Zn) cell can be estimated from the Gibbs free energy change in the electrochemical reaction according to:

E°=−ΔγG°/(nₑF)

where nₑ is the number of electrons transferred in the balanced half reactions, and F is Faraday's constant.

However, the voltage can be determined more conveniently using a potential table for the two half cells involved. To determine the voltage using a potential table, the first step is to identify the two metals and their ions reacting in the cell, and then look up the standard electrode potential, E°, in volts, for each of the two half reactions. The standard potential of the cell is equal to the more positive E° value minus the more negative E° value. For example, to calculate the standard potential with solutions comprising CuSO4 and Zinc sulfate (ZnSO4), copper and zinc's half reactions are used:

Cu2++2 e− [symbol] Cu E°=+0.34 V

Zn2++2 e− [symbol] Zn E°=−0.76 V

Thus, the overall reaction is:

Cu2++Zn [symbol] Cu+Zn2+

The standard potential for the reaction is then +0.34 V−(−0.76 V)=1.10 V. In such embodiments, the Zinc metal is more strongly reducing than copper metal because the standard (reduction) potential for zinc is more negative than that of copper. Thus, zinc metal will lose electrons to copper ions and develop a positive electrical charge (the polarity of the cell).

In some embodiments, the sensorial battery 130 is a zinc, copper battery. Other example sensorial batteries than may be employed within the described wetness monitoring system include, but are not limited to, an alkaline battery, an aluminum-air battery, a Bunsen cell, a chromic acid cell, a Clark cell, a Daniell cell, a Galvanic cell, a Grove cell, a Leclanché cell, a magnesium battery, a mercury battery, a nickel oxyhydroxide battery, a paper battery, a silver-oxide battery, a Voltaic pile, a zinc-air battery, a zinc-carbon battery, and a zinc chloride battery.

FIG. 2 depicts an example wetness monitoring system 200 that can be employed to execute implementations of the present disclosure. The example system 200 includes smart diapers 202, computing devices 212, 214, 216, a back-end system 230, and a network 210. In some embodiments, the smart diapers 202 are substantially similar to the smart diaper 100 depicted in FIG. 1A.

In some embodiments, the example system 200 is deployed within a hospital or health care center. The described wetness monitoring system 200 may be employed to, for example, provide real-real time wetness status for the smart diapers 202 via the computing devices 212, 214, 216 to the users 222, 224, and 226 (e.g., caretaker, staff, administrators, and so forth). In such examples, the users 212, 214, 216 may provide new smart diapers 202 to the wearers 204 (e.g., patients, children, and so forth). These wearers 204 may be in various rooms or otherwise dispersed through the facility to which the example system 200 is deployed.

In some embodiments, the network 210 includes a local area network (LAN), a wide area network (WAN), the Internet, or a combination thereof, and connects web sites, devices and systems (e.g., the smart diapers 202, the computing devices 212, 214, and 216, and the back-end system 230). In some embodiments, the network 210 includes the Internet, an internet, or an extranet. In some embodiments, the network 210 includes a telecommunication network or data network. In some embodiments, the network 210 can be accessed over a wired or a wireless communications link, such as a network gateway. For example, the transmitter embedded in each smart diaper 202 may broadcast a signal that is received by a network gateway or mobile computing devices (e.g., the smartphone device 212 and the tablet device 216), can use a cellular network to access the network 210. In some embodiments, the network 210 includes a network of physical objects (or Internet of things) with mesh and start topological structures (e.g., Narrowband IoT (NBIOT), Long Range (LoRa, ZigBee, general package radio service (GPRS), and Long-Term Evolution (LTE) category MI (Cat1)). In some embodiments, protocols can be adopted within the network 210 for specific applications and environments. For example, mesh topology can be used for IOTs of a smaller scale. Moreover, a mesh topology generally supports longer distance data transmission and can be used for IOTs of a larger scale.

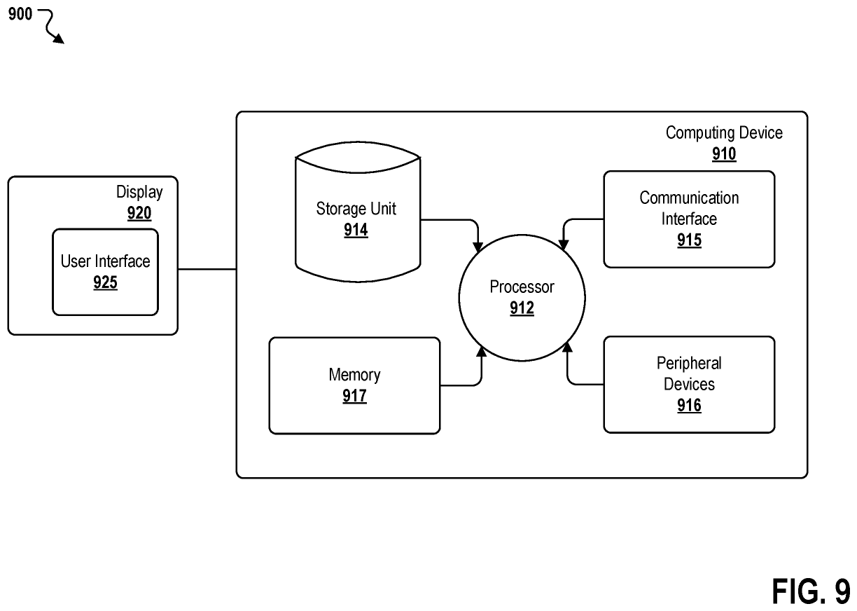

In the depicted example system 200, the back-end system 230 includes at least one server device 232 and at least one data store 234. In some embodiments, the device 232 is sustainably similar to computing device 910 depicted in FIG. 9. In some embodiments, back-end system 230 may include server-class hardware type devices. In some embodiments, the server device 232 is a server-class hardware type device. In some embodiments, back-end system 230 includes computer systems using clustered computers and components to act as a single pool of seamless resources when accessed through the network 210. For example, such implementations may be used in data center, cloud computing, storage area network (SAN), and network attached storage (NAS) applications. In some embodiments, back-end system 230 is deployed using a virtual machine(s).

In some embodiments, the data store 234 is a repository for persistently storing and managing collections of data. Example data store that may be employed within the described wetness monitoring system include data repositories, such as a database as well as simpler store types, such as files, emails, and so forth. In some embodiments, the data store 234 includes a database, which may include a series of bytes or an organized collection of data that is managed by a database management system (DBMS). In some embodiments, the data store 234 is provided via a distributed ledger (e.g., a blockchain).

In some embodiments, the computing devices 212, 214, and 216 are sustainably similar to computing device 910 depicted in FIG. 9. The computing devices 212, 214, and 216 may each include any appropriate type of computing device such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In the depicted example, the computing device 212 is a smartphone, the computing device 214 is a tablet-computing device, and the computing device 216 is a desktop computing device. Three user computing devices 212, 214, and 216 and four smart diapers 202 are depicted in FIG. 2 for simplicity. It is contemplated, however, that implementations of the present disclosure can be realized with any of the appropriate computing devices or smart diapers, such as those mentioned previously. Moreover, implementations of the present disclosure can employ any number of devices as required.

In some embodiments, the at least one server system 232 hosts one or more computer-implemented services provided by the described wetness monitoring system 200 that users 222, 224, and 226 can interact with using the respective computing devices 212, 214, and 216. In some examples, the users 222, 224, and 226 interact with the wetness monitoring system through a graphical user interface (GUI) or application that is installed and executing on their respective computing devices 212, 214, and 216. For example, when a smart diaper 202 is put on a wearer 204 (or the wearer puts it on), the users 222, 224, and 226 may employ their respective user devices 212, 214, 216 to scan a barcode associated with the smart diaper 202 and associate the smart diaper 202 (e.g., via a unique identifier) with the wearer 204 (e.g., a staff member may provide location information such as a room number for the patient or utilize a patient ID). The information associating the wearer 204 with the smart diaper 202 may be provided to the at least one server system 232 by the user devices 212, 214 or 216 for storage in the data store 234.

In some embodiments, once powered, a transmitter that is embedded in one of the smart diapers 202 transmits a signal (e.g., a beacon type broadcast via, for example iBeacon) to the network 210 via a wireless network gateway. In some embodiments, the network gateway converts the wireless beacon to a specific format and provides the information to the back-end system 230 or directly to the computing devices 212, 214, and 216. In some embodiments, a base station or network server may be employed to provide the broadcast information to the end point. In some embodiments, the wireless gateway includes multiple antennas for receiving the beacons for the transmitters embedded in the smart diapers 202 and broadcasting to the network 110.

In some embodiments, the computing devices 212, 214, and 216 may receive the information (e.g., a unique identifier associated with one of the smart diapers 202) from the back-end system 230 when broadcasted by a transmitter. In some embodiments, the computing devices 212, 214, and 216 may directly receive the beacons broadcast from each transmitter. In some embodiments, when the computing devices 212, 214, and 216 receive the information broadcast from a transmitter, the wearer 204 associated with the respective smart diaper 202 is identified and a notification is provided to the respective user that the wearer(s) has a wet smart diaper. In some embodiments, the computing devices 212, 214, and 216 are configured to emit a sound, flash a light, or display information (e.g., location data, room number, patient data, and so forth) regarding the smart diaper associated with the received unique identifier to provide an indication that the smart diaper 202 is wet and the wearer 204 requires a new smart diaper 202. In some embodiments, the back-end system 230 or the computing devices 212, 214, and 216 provide updates via a screen in the facility. For example, real-time status for the smart diapers 202 can be shown on the screen for the staff to see.

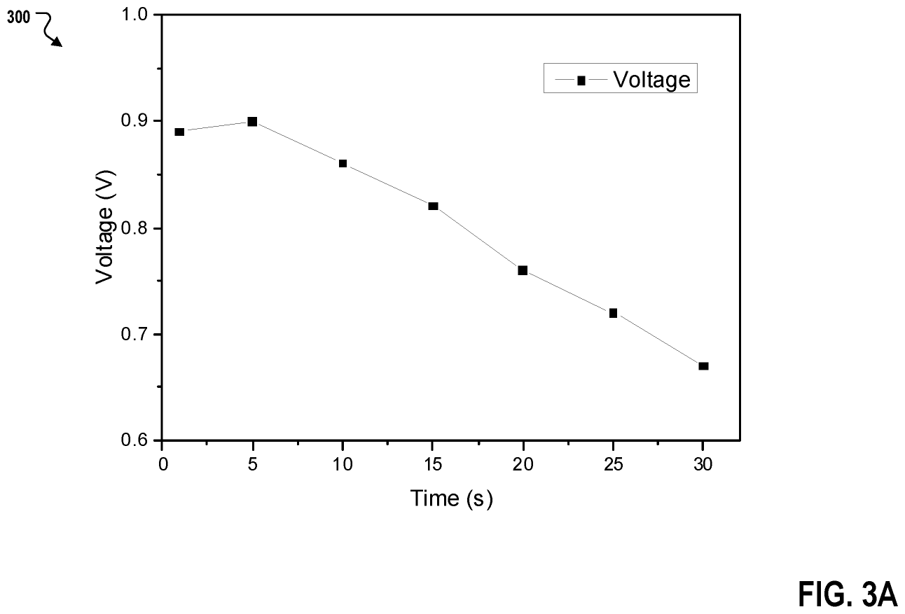

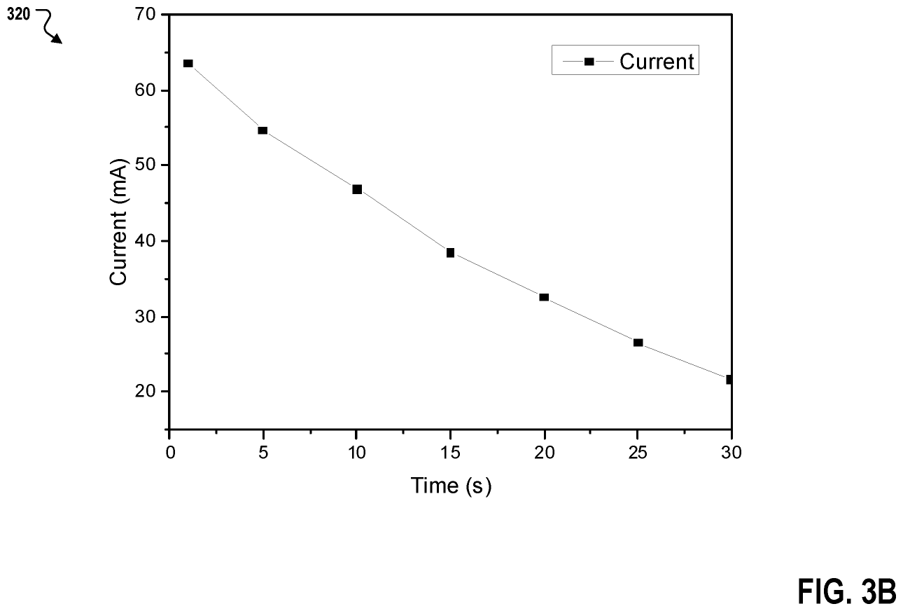

FIG. 3A depicts a graph 300 plotting time against voltage detected after an amount of urine is added to a sensorial battery, such as the sensorial battery 130 depicted in FIGS. 1A and 1B. FIG. 3B depicts a graph 320 plotting time against the current drop detected after an amount of urine is added to the sensorial battery. As shown if FIG. 3A, the measured sensorial battery produced an initial voltage of 0.89 Volts (V), which dropped gradually as the reaction continued. A similar result can be seen in the plot of current in FIG. 3B that shows the largest value of the current was measured at 64 milli Ampere (mA), which dropped gradually to 20 mA in approximately 30 seconds.

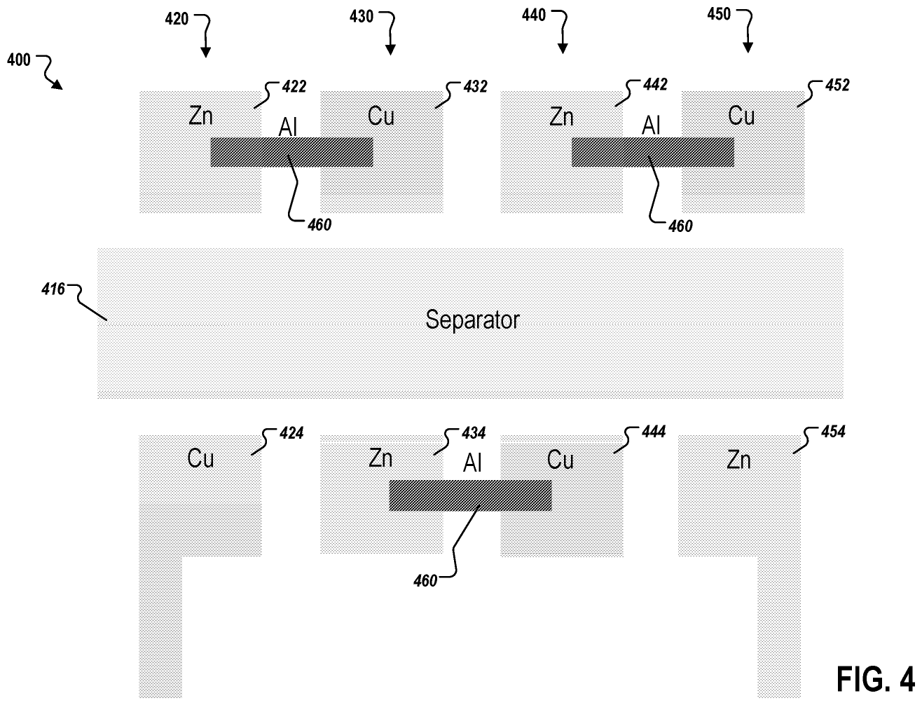

FIG. 4 depicts a perspective view of a sensorial battery 400 comprised of multiple batteries (e.g., pairs of electrodes) that can be employed within the described wetness monitoring system. As depicted, the sensorial battery 400 comprises a separator 416 and four batteries 420, 430, 440, and 450, that are connected via connectors 460. The depicted embodiment shows four batteries as an example; however, any number of batteries can be employed based on the amount of power required (e.g., for powering a transmitter).

The separator 416 is substantially similar to the separator 136 describe above. In some embodiments, the separator 416 comprises a piece of flexible material and is used as the substrate to support the batteries 420, 430, 440, and 450. In the depicted embodiment, pairs of electrodes, 422 and 424, 432 and 434, 442 and 444, and 452 and 454, are arranged in parallel at an interval. In the depicted embodiment, first two electrodes 422 and 424 and the last two electrodes 452 and 454 are connected via connectors 460. In some embodiments, the connectors a comprised of metal (e.g., aluminum) foil or other conductive materials.

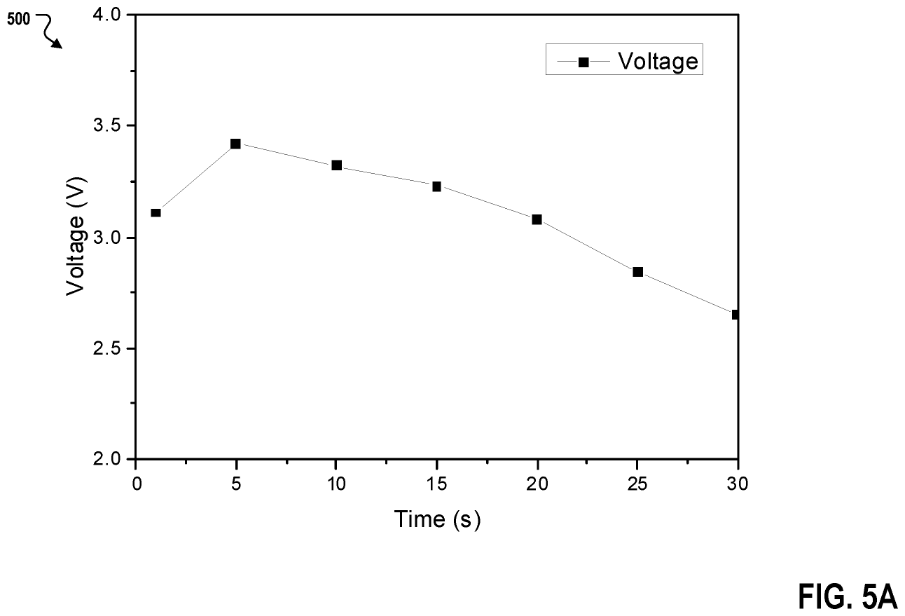

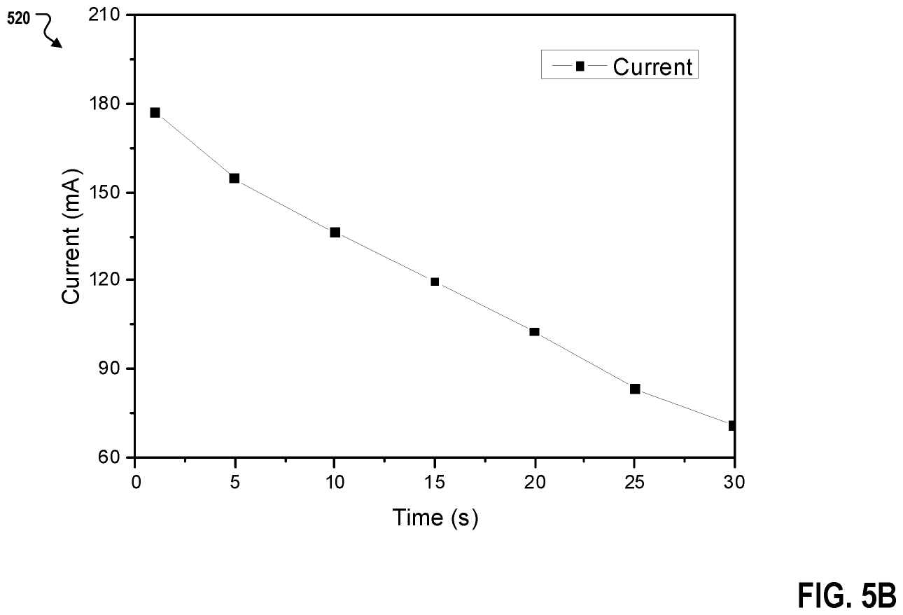

FIG. 5A depicts a graph 500 plotting time against voltage detected after an amount of urine is added to a sensorial battery comprising four batteries, such as the sensorial battery 400 depicted in FIG. 4. FIG. 5B depicts a graph 520 plotting time against the current drop detected after an amount of urine is added to the sensorial battery comprising four batteries. As shown in FIG. 5A, voltage increases after adding urine, which may attribute to fully infiltration at the initial process. After five seconds, the voltage gradually drops down to 2.6 V as the electrode material deplete in the reaction. As shown in FIG. 5B, the current is 178 mA after urine is added in the battery system and then drops down to 70 mA in approximately 30 seconds.

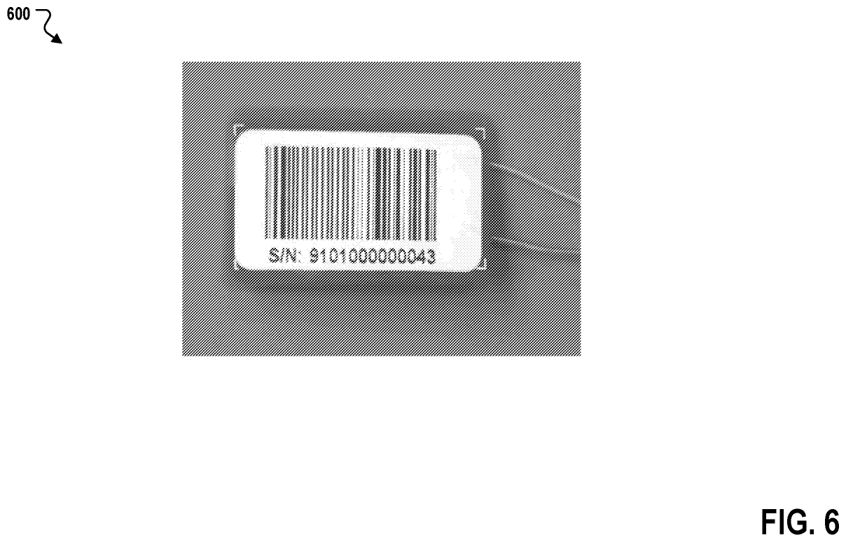

FIG. 6 depicts an embodiment of a barcode 600 that can be employed in the described wetness monitoring system. In some embodiments, the barcode 600 is deposed on a smart tag, such as smart tag 110 depicted in FIG. 1A, to associate a unique identifier with the respective transmitter.

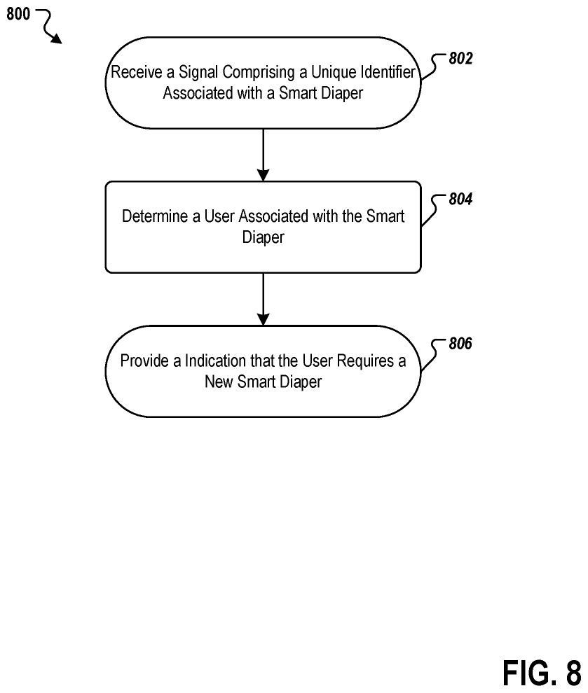

FIG. 8 depicts a flow diagram of an example process 800 for monitoring the wetness of a smart diaper. In some embodiment, the process is executed by one or more processors such as described in FIG. 9. For clarity of presentation, the description that follows generally describes the process 800 in the context of FIGS. 1-7 and 9. However, it will be understood that the process 800 may be performed, for example, by any other suitable system or a combination of systems as appropriate.

At 802, a signal is received from a transmitter embedded in a smart diaper. In some embodiments, the transmitter is powered through a sensorial battery configured to provide electric current to the transmitter in the presence of water or urine. In some embodiments, the signal comprises a unique identifier associated with the smart diaper. In some embodiments, before receiving the signal, a barcode comprising the unique identifier is scanned via a scanning device. In some embodiments, user data regarding the user and instructions to associate the user with the smart diaper is received via an input device. In some embodiments, scanning device comprises a camera. In some embodiments, the input device comprises a touch screen. In some embodiments, the association between the smart diaper and the user is persisted (e.g., in a datastore). From 802, the process 800 proceeds to 804.

At 804, a user associated with the smart diaper is determined based on the unique identifier. From 804, the process 800 proceeds to 806.

At 806, an indication that a user requires a new smart diaper is provided via a user interface. From 806, the process 800 ends.

### Processing Devices and Processors

In some embodiments, the platforms, systems, media, and methods described herein include a computer, or use of the same. In further embodiments, the computer includes one or more hardware central processing units (CPUs) or general-purpose graphics processing units (GPGPUs) that carry out the device's functions. In still further embodiments, the computer comprises an operating system configured to perform executable instructions. In some embodiments, the computer is optionally connected a computer network. In further embodiments, the computer is optionally connected to the Internet such that it accesses the World Wide Web. In still further embodiments, the computer is optionally connected to a cloud computing infrastructure. In other embodiments, the computer is optionally connected to an intranet. In other embodiments, the computer is optionally connected to a data storage device.

In accordance with the description herein, suitable computers include, by way of non-limiting examples, server computers, desktop computers, laptop computers, notebook computers, sub-notebook computers, netbook computers, netpad computers, handheld computers, Internet appliances, mobile smartphones, tablet computers, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the computer includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the computer is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, compact disc read-only memories (CD-ROMs), digital versatile discs (DVDs), flash memory devices, magnetic disk drives, magnetic tapes drives, optical disk drives, and cloud computing-based storage. In further embodiments, the storage and/or memory device is a combination of devices such as those disclosed herein.

In some embodiments, the computer includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the computer, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the computer includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

Computer systems are provided herein that can be used to implement methods or systems of the disclosure. FIG. 9 depicts an example 900 of a computer system 910 that can be programmed or otherwise configured to implement methods or systems of the present disclosure. For example, the computing device 910 can be programmed or otherwise configured to provide an indication that a smart diaper associated with a user has become wet.

In the depicted embodiment, the computing device 910 includes a CPU (also “processor” and “computer processor” herein) 912, which is optionally a single core, a multi core processor, or a plurality of processors for parallel processing. The computing device 910 also includes memory or memory location 917 (e.g., random-access memory, read-only memory, flash memory), electronic storage unit 914 (e.g., hard disk), communication interface 915 (e.g., network adapter) for communicating with one or more other systems, and peripheral devices 916, such as cache, other memory, data storage and/or electronic display adapters. The memory 917, storage unit 914, interface 915 and peripheral devices 916 are in communication with the CPU 912 through a communication bus (solid lines), such as a motherboard. The storage unit 914 comprises a data storage unit (or data repository) for storing data. The computing device 910 is optionally operatively coupled to a computer network, such as the network 210 depicted in FIG. 2, with the aid of the communication interface 915. In some embodiments, the computing device 910 is configured as a node within a peer-to-peer network.

In some embodiments, the CPU 912 can execute a sequence of machine-readable instructions, which can be embodied in a program or software. The instructions may be stored in a memory location, such as the memory 917. The instructions can be directed to the CPU 912, which can subsequently program or otherwise configure the CPU 912 to implement methods of the present disclosure. Examples of operations performed by the CPU 912 can include fetch, decode, execute, and write back. In some embodiments, the CPU 912 is part of a circuit, such as an integrated circuit. One or more other components of the computing device 910 can be optionally included in the circuit. In some embodiments, the circuit is an application specific integrated circuit (ASIC) or a FPGA.

In some embodiments, the storage unit 914 stores files, such as drivers, libraries and saved programs. In some embodiments, the storage unit 914 stores user data, e.g., user preferences and user programs. In some embodiments, the computing device 910 includes one or more additional data storage units that are external, such as located on a remote server that is in communication through an intranet or the internet.

In some embodiments, the computing device 910 communicates with one or more remote computer systems through a network. For instance, the computing device 910 can communicate with a remote computer system. Examples of remote computer systems include personal computers (e.g., portable PC), slate or tablet PCs (e.g., Apple® iPad, Samsung Galaxy Tab, etc.), smartphones (e.g., Apple® iphone, Android-enabled device, Blackberry®, etc.), or personal digital assistants. In some embodiments, a user can access the computing device 910 via a network.

In some embodiments, methods as described herein are implemented by way of machine (e.g., computer processor) executable code stored on an electronic storage location of the computing device 910, such as, for example, on the memory 917 or the electronic storage unit 914. In some embodiments, the CPU 912 is adapted to execute the code. In some embodiments, the machine executable or machine-readable code is provided in the form of software. In some embodiments, during use, the code is executed by the CPU 912. In some embodiments, the code is retrieved from the storage unit 914 and stored on the memory 917 for ready access by the CPU 912. In some situations, the electronic storage unit 914 is precluded, and machine-executable instructions are stored on the memory 917. In some embodiments, the code is pre-compiled. In some embodiments, the code is compiled during runtime. The code can be supplied in a programming language that can be selected to enable the code to execute in a pre-compiled or as-compiled fashion.

In some embodiments, the computing device 910 can include or be in communication with an electronic display 920. In some embodiments, the electronic display 920 provides a user interface (UI) 925.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computer. In further embodiments, a computer readable storage medium is a tangible component of a computer. In still further embodiments, a computer readable storage medium is optionally removable from a computer. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the computer's CPU, written to perform a specified task. Computer readable instructions may be implemented as program modules, such as functions, objects, API, data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and extensible Markup Language (XML) database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or XML. In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe Flash®, HTML 5, Apple® QuickTime®, Microsoft® Silverlight®, Java™, and Unity®.

### Mobile Application

In some embodiments, a computer program includes a mobile application provided to a mobile computer. In some embodiments, the mobile application is provided to a mobile computer at the time it is manufactured. In other embodiments, the mobile application is provided to a mobile computer via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm OS SDK, Symbian SDK, webOS SDK, and Windows Mobile SDK.

Those of skill in the art will recognize that several commercial forums are available for distribution of mobile applications including, by way of non-limiting examples, Apple® App Store, Google Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Data Stores

In some embodiments, the platforms, systems, media, and methods disclosed herein include one or more data stores. In view of the disclosure provided herein, those of skill in the art will recognize that data stores are repositories for persistently storing and managing collections of data. Types of data stores repositories include, for example, databases and simpler store types, or use of the same. Simpler store types include files, emails, and so forth. In some embodiments, a database is a series of bytes that is managed by a DBMS. Many databases are suitable for receiving various types of data, such as weather, maritime, environmental, civil, governmental, or military data. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In some embodiments, a database is web-based. In some embodiments, a database is cloud computing based. In some embodiments, a database is based on one or more local computer storage devices.

While preferred embodiments of the present disclosure have been shown and described herein, it will be obvious to those skilled in the art that such embodiments are provided by way of example only. Numerous variations, changes, and substitutions will now occur to those skilled in the art without departing from the described system. It should be understood that various alternatives to the embodiments described herein may be employed in practicing the described system.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Moreover, the separation or integration of various system modules and components in the implementations described earlier should not be understood as requiring such separation or integration in all implementations, and it should be understood that the described components and systems can generally be integrated together in a single product or packaged into multiple products. Accordingly, the earlier description of example embodiments does not define or constrain this disclosure. Other changes, substitutions, and alterations are also possible without departing from the spirit and scope of this disclosure.

Thus, the disclosure provides, among other things, a wetness monitoring system to monitor the wetness of a smart diaper associated (e.g., being worn by) with a user. Various features and advantages of the disclosure are set forth in the following claims.

What is claimed is:

1. A wetness monitoring system comprising:
a smart diaper comprising:
a sensorial battery comprising at least two electrodes; and
a transmitter, wherein the sensorial battery is configured to provide electric current to the transmitter in the presence of water or urine, and wherein the transmitter is configured to emit a signal comprising a unique identifier when the electric current is received from the sensorial battery; and
a computing device comprising:
a receiver;
a user interface;
one or more processors; and
a computer-readable storage device coupled to the one or more processors and having instructions stored thereon which, when executed by the one or more processors, cause the one or more processors to perform operations comprising:
receiving, via the receiver, the signal emitted by the transmitter, and
providing, via the user interface, an indication that the smart diaper is wet.

2. The wetness monitoring system of claim 1, wherein the sensorial battery is embedded on an interior surface of the smart diaper.

3. The wetness monitoring system of claim 1, wherein the smart diaper comprises an absorption layer, and wherein the sensorial battery is embedded within between 2 and 20 millimeters of the absorption layer of the diaper.

4. The wetness monitoring system of claim 1, wherein the sensorial battery comprises an electrochemical cell.

5. The wetness monitoring system of claim 4, wherein the sensorial battery, when wet, derives electrical energy from spontaneous redox reactions taking place within the electrochemical cell.

6. The wetness monitoring system of claim 1, wherein the at least two electrodes comprise a positive and a negative electrode, wherein the sensorial battery comprises a separator that mechanically isolates the positive and negative electrodes, and wherein the separator comprises dried filter paper that has absorbed a metallic salt solution.

7. The wetness monitoring system of claim 6, wherein the separator is configured to release the dried metallic salt solution in the presence of water or urine as an ionized liquid to electrically connect the at least two electrodes generating the electric current via a potential difference and charge flow.

8. The wetness monitoring system of claim 1, wherein at least one of the electrodes comprises copper, and wherein at least one of the electrodes comprise zinc.

9. The wetness monitoring system of claim 1, wherein the signal is emitted as a wireless beacon by the transmitter.

10. The wetness monitoring system of claim 1, wherein the computing device comprises a smart phone or smart tablet.

11. The wetness monitoring system of claim 1, wherein the operations comprise:
determining, based on the unique identifier, a user associated with the smart diaper, and
providing, via the user interface, an indication that user requires a new smart diaper.

12. The wetness monitoring system of claim 11, wherein the operations comprise:
before determining the user, receiving information associating the smart diaper to the user via the unique identifier; and
persisting the association between the smart diaper and the user.

13. A smart diaper comprising:
a smart tag embedded in an interior surface of a diaper, the smart tag comprising:
a transmitter; and
a sensorial battery comprising at least one anode, at least one cathode, and a separator comprising dried filter paper that has absorbed a metallic salt solution, the separator mechanically isolating the at least one anode from the at least one cathode, wherein the separator is configured to release the dried metallic salt solution in the presence of water or urine as an ionized liquid to electrically connect the at least one anode and the at least one cathode generating electric current, wherein the sensorial battery is configured to provide the electric current to the transmitter, and wherein the transmitter is configured to emit a signal comprising a unique identifier when the electric current is received.

14. The smart diaper of claim 13, wherein the smart diaper comprises an absorption layer, and wherein the sensorial battery is embedded within between 2 and 20 millimeters of the absorption layer of the diaper.

15. The smart diaper of claim 13, wherein the at least one cathode comprises copper, and wherein the at least one anode comprises zinc.

16. The smart diaper of claim 13, wherein the sensorial battery comprises an alkaline battery, an aluminum-air battery, a Bunsen cell, a chromic acid cell, a Clark cell, a Daniell cell, a Galvanic cell, a Grove cell, a Leclanché cell, a magnesium battery, a mercury battery, a nickel oxyhydroxide battery, a paper battery, a silver-oxide battery, a Voltaic pile, a zinc-air battery, a zinc-carbon battery, or a zinc chloride battery.

17. The smart diaper of claim 13, wherein the signal is emitted as a wireless beacon by the transmitter.

18. A computer-implemented method for monitoring the wetness of a smart diaper, the method being executed by one or more processors and comprising:
receiving, from a transmitter embedded in a smart diaper, a signal comprising a unique identifier associated with the smart diaper, the transmitter powered through a sensorial battery configured to provide electric current to the transmitter in the presence of water or urine;
determining, based on the unique identifier, a user associated with the smart diaper, and
providing, via a user interface, an indication that a user requires a new smart diaper.

19. The method of claim 18, further comprising:
before receiving the signal, scanning, via a scanning device, a barcode comprising the unique identifier;
receiving, via an input device, user data regarding the user and instructions to associate the user with the smart diaper; and
persisting the association between the smart diaper and the user.

20. The method of claim 19, wherein the scanning device comprises a camera, and wherein the input device comprises a touch screen.
